(19) United States
(12) Patent Application Publication
(10) Pub. No.: US 2016/0260216 A1
(43) Pub. Date: Sep. 8, 2016

(54) SYSTEM AND METHOD FOR QUANTITATIVE ANALYSIS OF NUCLEAR MEDICINE BRAIN IMAGING

(71) Applicant: National Yang-Ming University, Taipei (TW)

(72) Inventors: Tung-Hsin Wu, Taipei (TW); Bang-Hung Yang, Taipei City (TW); Jhih-Shian Lee, New Taipei City (TW); Yu-Xiang Guan, Zhuangwei Township (TW)

(21) Appl. No.: 14/819,862

(22) Filed: Aug. 6, 2015

(30) Foreign Application Priority Data
Mar. 6, 2015 (TW) 104107190

(51) Int. Cl. G06T 7/00; A61B 5/00

(52) U.S. Cl. CPC G06T 7/0016; G06T 7/0081; A61B 5/4082; A61B 5/743; G06T 2207/20021; G06T 2207/20144; G06T 2207/30016

(57) ABSTRACT

The invention provides a method for quantitative analysis of nuclear medicine brain imaging. The method comprises retrieving a target image, wherein the target image is a brain image as a nuclear medicine image produced from a radiopharmaceutical, then matching the position of a space axis and the size of a voxel of the target image to a standard template by an affine transformation, wherein the standard template comprises a relative position of striatum. The method further comprises selecting the striatum from the target image according to the relative position of the striatum in the standard template and calculating an average value of the striatum, dividing the striatum from the target image and calculating a background value based on a remainder pixel value of the target image, and a specific uptake ratio is calculated based on the average value of the striatum and the background value.

### CROSS-REFERENCE TO RELATED APPLICATIONS

This Non-provisional application claims priority under 35 U.S.C. §119(a) on Patent Application No(s). [104107190] filed in Taiwan, Republic of China [Mar. 6, 2015], the entire contents of which are hereby incorporated by reference.

### FIELD OF THE INVENTION

The present invention provides a system and a method about the nuclear medicine imaging process, and more particularly, to a system and a method for quantitative analysis of nuclear medicine imaging.

### BACKGROUND OF THE INVENTION

Enabling earlier diagnosis of Parkinson's disease (PD) is a goal of the nuclear medicine test. Parkinson's disease is primarily caused by a progressive decrease of dopaminergic neurons in the nigrostriatal pathway and is characterized by an insidious onset of motor symptoms such as rigidity, tremor, and bradykinesia. Recent studies showed that an unexpectedly high rate of misdiagnosis occurred if the diagnosis was based on only the clinical diagnostic criteria. With the development of computed tomography (CT) and magnetic resonance imaging (MRI), the imaging techniques is more specific to confirm the location of damage in brain injured patients. The measurement of the electrical signals on the scalp, arising from the synchronous firing of the neurons in response to a stimulus, known as electroencephalography (EEG), opened up new possibilities to study brain function in normal subjects. It was the advent of the functional imaging modalities of single photon emission computed tomography (SPECT) that led to a new era in the study of brain function. The imaging modalities of single photon emission computed tomography involves the use of radioactive nuclides either from natural or synthetic sources. Their strength is in the fact that, since the radioactivity is introduced, they can be used in tracer studies where a radiopharmaceutical is selectively absorbed in a region of the brain. Single photon emission computed tomography has enabled noninvasive, in vivo visualization of the progression of striatal neuronal function in Parkinson's disease patients. Therefore, the single photon emission computed tomography has become a great help in the diagnostic of Parkinson's disease.

At present, the diagnosis of Parkinson's disease still depends on clinical criteria. Parkinson's disease is a common disorder, and the diagnosis of Parkinson's disease is clinical and relies on the presence of characteristic motor symptoms. The accuracy of the clinical diagnosis of Parkinson's disease is still limited. Brain imaging is performed using radiopharmaceuticals by single photon emission computed tomography. The expert will determine if the scan follows the pattern of Parkinson's disease. It is important to keep in mind that the reading of SPECT images should be performed only by experienced neurologists who have executed a large volume of Parkinson's disease scans, because experience is important in accurately reading these imaging results. It takes an expert to read these scans and figure out if the changes are due to normal aging or due to disease or selects the striatum region for using semi quantitative analysis to observe dopaminergic neurons in the brain image. However, it can lead to issues in misdiagnosis, time-consuming, reproducibility and personal subjectivity.

Brain imaging is performed using radiopharmaceuticals by single photon emission computed tomography. Radiopharmaceuticals are used in the field of nuclear medicine as radioactive tracers in medical imaging and in detection various cranial nerve diseases such as stroke, Parkinson's disease, Alzheimer's disease, epilepsy, and psychiatric disorders. However, the amount of the tracer absorption (uptake) that occurs (which influences the sensitivity of the gamma camera), and the clarity of images (that is the spatial resolution) it produces. Many radiopharmaceuticals are usually able to focus on just one particular part of the body, which can affect the contrast ratio of the brain image and cause an error of a spatial transformation. These facts imply that the accuracy of diagnosing cranial nerve disease needs improvement.

### SUMMARY OF THE INVENTION

The present invention provides a system and a method about nuclear medicine imaging process, and more particularly, to a system and a method for quantitative analysis of nuclear medicine imaging based on the specific uptake ratio and the asymmetry index.

In an embodiment of the invention, the present invention provides the method for quantitative analysis of brain nuclear medicine imaging. The method comprises retrieving a target image, wherein the target image is a nuclear medicine image of brain produced from a radiopharmaceutical, then mapping a coordinate space and a voxel shape of the target image to a standard brain template by an affine transformation, wherein the standard brain template comprises a striatum relative position. The method further comprises determining a striatum region from the target image according to the striatum relative position in the standard brain template and calculating an average pixel value of the striatum region, dividing the striatum region from the target image, and calculating a background value based on a pixel value of a remainder region from the target image, and a specific uptake ratio is calculated based on the average pixel value of the striatum region and the background value.

In another embodiment of the invention, the present invention provides the system for quantitative analysis of brain nuclear medicine imaging. The system comprises an image capturing unit configured to capture a target image, wherein the target image is a brain image as a nuclear medicine image produced of brain from a radiopharmaceutical. A processing unit configured to map a coordinate space and a voxel shape of the target image to a standard brain template by an affine transformation, wherein the standard brain template comprises a striatum relative position, then extract a striatum region from the target image according to the striatum relative position in the standard brain template and calculate an average pixel value of the striatum region, dividing the striatum region from the target image, and calculating a background value based on a pixel value of a remainder region from the target image. A computing unit configured to calculate a first specific uptake ratio based on the average pixel value of the striatum region and the background value.

### BRIEF DESCRIPTION OF THE DRAWINGS

FIG. 1 illustrates an embodiment about a block diagram of the system for quantitative analysis of nuclear medicine brain imaging;

FIG. 2 illustrates an embodiment about a nuclear medicine image processed by stereotactic normalization ;

FIG. 3 illustrates an embodiment about a striatum relative position of the standard brain template;

FIG. 4 illustrates an embodiment about a striatum region extracted from the target image according to the striatum relative position ;

FIG. 5 illustrates an embodiment about a nuclear medicine image without the striatum region ;

FIG. 6 illustrates an embodiment about a reduced type of the striatum;

FIG. 7 illustrates an embodiment about a window of the system to process the nuclear medicine image; and

FIG. 8 illustrates an embodiment about a flowchart of the method for quantitative analysis of nuclear medicine brain imaging.

### DETAILED DESCRIPTION OF THE INVENTION

The present invention discloses a system and a method for quantitative analysis of nuclear medicine imaging. It is understood that the method provides merely an example of the many different types of functional arraignments that may be employed to implement the operation of the various components of a system for quantitative analysis of nuclear medicine imaging, a computer system connected to a scanner, a multiprocessor computing device, and so forth.

The execution steps of the present invention may include application specific software which may store in any portion or component of the memory including, for example, random access memory (RAM), read-only memory (ROM), hard drive, solid-state drive, magneto optical (MO), IC chip, USB flash drive, memory card, optical disc such as compact disc (CD) or digital versatile disc (DVD), floppy disk, magnetic tape, or other memory components.

For embodiments, the system comprises a display device, a processing unit, a memory, an input device and a storage medium. The input device used to provide data such as image, text or control signals to an information processing system such as a computer or other information appliance. In accordance with some embodiments, the storage medium such as, by way of example and without limitation, a hard drive, an optical device or a remote database server coupled to a network, and stores software programs. The memory typically is the process in which information is encoded, stored, and retrieved etc. The processing unit performs data calculations, data comparisons, and data copying. The display device is an output device that visually conveys text, graphics, and video information. Information shown on the display device is called soft copy because the information exists electronically and is displayed for a temporary period of time. Display devices include CRT monitors, LCD monitors and displays, gas plasma monitors, and televisions. In accordance with such embodiments of present invention, the software programs are stored in the memory and executed by the processing unit when the computer system executes the method for quantitative analysis of nuclear medicine imaging. Finally, information provided by the processing unit, presented on the display device or stored in the storage medium.

The present invention provides a system and a method for quantitative analysis of nuclear medicine imaging according to the differences in a specific uptake ratio of the striatum region on a three-dimensional surface for automatically determining Parkinson's disease stage and provides the quantification of the specific uptake ratio to improve outcomes through early diagnosis of Parkinson's disease.

Radiopharmaceuticals are used in the field of nuclear medicine as radioactive tracers in medical imaging and in therapy for many diseases. The amount of the tracer absorption (uptake) that occurs (which influences the sensitivity of the gamma camera), and the clarity of images it produces. Many radiopharmaceuticals are usually able to focus on just one particular part of the body, which can make treatment a lot more effective. The present invention provides a method and system for using Tc-99m TRODAT-1 to help rule out the possibility of Parkinson's disease as early as possible by diagnosis the striatum.

The method and system not only provides the reference values for structural and functional abnormalities in the brain also automatically to determine a putamen and a caudate nucleus of the striatum region to avoid an error from the manual section can lead to misdiagnosis. The present invention also provides the specific uptake ration and the asymmetry index to diagnose the loss of dopamine neurons in Parkinson's disease.

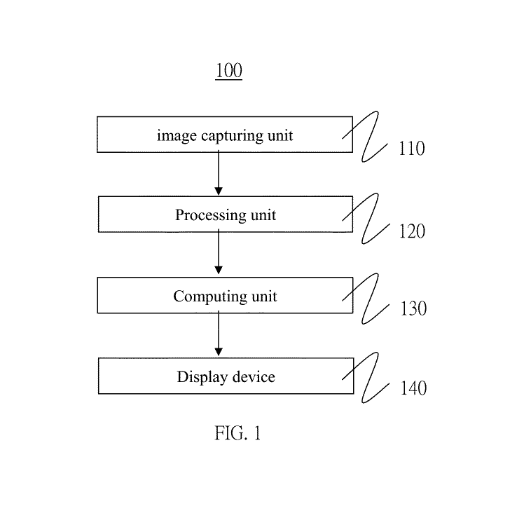

Please refer FIG. 1, FIG. 1 is a block diagram of the system for quantitative analysis of nuclear medicine brain imaging. The system 100 comprises an image capturing unit 110, a processing unit 120, a computing unit 130 and a display device 140.

For most diagnostic studies in the functional of brain imaging, the present invention provides the radioactive tracer for example, Tc-99m TRODAT-1 is administered to a patient by intravenous injection. The image capturing unit 110 is configured to scan the brain from multiple angles by a scanner. The scanner was rotated 180 degrees or 360 degrees multiple 2-D images (also called projections), from multiple angles. The image capturing unit 110 collects the information emitted by the gamma rays and translates them into multiple 2-D images. The image capturing unit 110 is then used to apply an algorithm to the multiple projections form a target image as a 3D image, wherein the target image is a scan image

Please refer FIG. 2, FIG. 2 illustrates a target image is processed by stereotactic normalization. The processing unit 120 processes the target image 200 with stereotactic normalization by statistical parametric mapping (SPM), and then the processing unit 120 processes the target image 200 with affine transformation including translation, zooms, rotations and shears. The processing unit 120 further applies a non-linear transformation to the target image 200 to obtain a deformed target image. The processing unit 120 (FIG. 1) maps the deformed target image onto a standard brain template that already conforms to a standard stereotactic space (e.g. an anatomical space) and a size of voxel (2×2×2 mm3) based on a minimized mean-squared difference between the deformed target image and the standard brain template. As depicted in FIG. 2 the target image 200 had processed by stereotactic normalization, which shows the radioactive tracer, for example Tc-99m TRODAT-1 used to track the distribution of a substance within the brain tissue.

The target image 200 had processed by stereotactic normalization to a talairach daemon space. The talairach daemon space is a 3-dimensional coordinate system of the human brain, which is used to map the location of brain structures independent from individual differences in the size and overall shape of the brain. It is common to use talairach coordinates in functional brain imaging studies and depicts the basic anatomical structures and stereotactic coordinates of brain regions.

The processing unit 120 (FIG. 1) is configured to determine the striatum region in the target image 200, wherein the striatum region is corresponding to a striatum relative position of the standard brain template. The processing unit 120 (FIG. 1) further calculates an average pixel value of the striatum region.

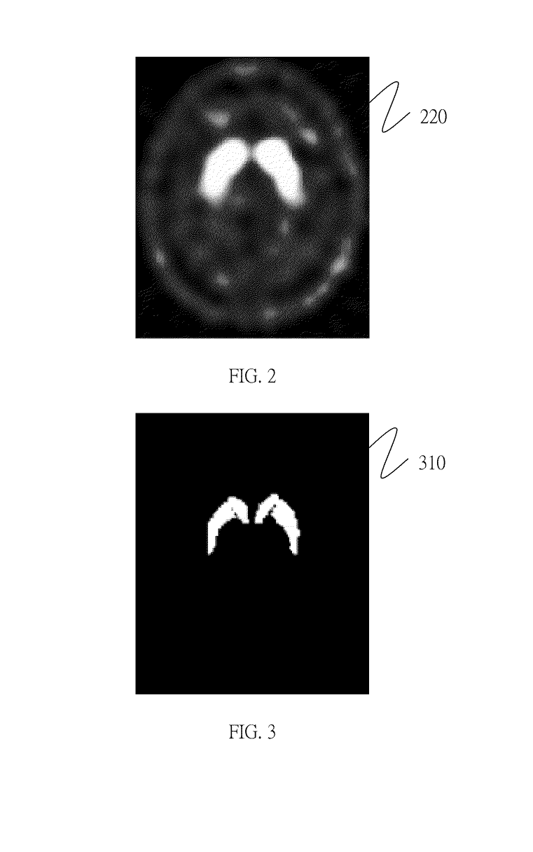

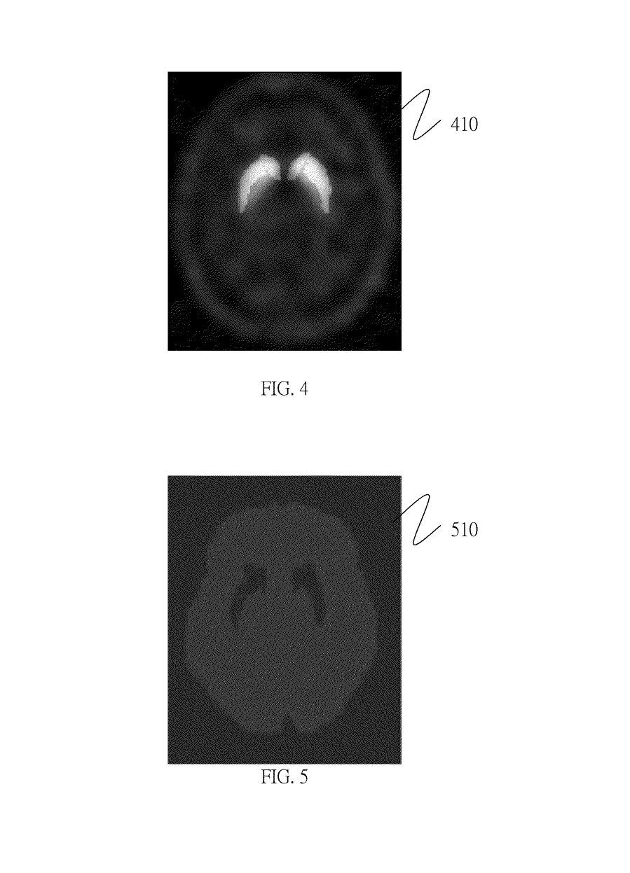

Please refer FIG. 2, FIG. 3 and FIG. 4. FIG. 3 illustrates an embodiment about the striatum region of a nuclear medicine image mapping with the standard brain template. FIG. 4 is a striatum region extracted from the target image according to the striatum relative position. The basal ganglia located deep in the cerebral cortex comprises multiple subcortical nuclei, of varied origin, in the brains of vertebrates, which are situated at the base of the forebrain. The basal ganglia include caudate nucleus, putamen, globus pallidus, and substantia nigra. The striatum is used here because of the striated appearance produced by the strands of gray matter passing through the internal capsule and connecting the caudate nucleus to the putamen of the lentiform nucleus, wherein the caudate nucleus and the putamen are functionally and histologically similar nuclei.

The processing unit 120 atomically determines the striatum region 410 which processed by stereotactic normalization in FIG. 4 based on comparing the striatum relative position 310 of the standard brain template in FIG. 3 with the target image 200 which processed by stereotactic normalization in FIG. 2. The striatum 310, 410 comprises the region of the caudate nucleus and the putamen.

For example, the processing unit 120 automatically determines a putamen and a caudate nucleus of the striatum region in the Tc-99m TRODAT-1 target image in FIG. 4 according to a relative position of the striatum which defined in the standard brain template in FIG. 3. The processing unit 120 (FIG. 1) further calculates the average pixel value of the striatum region 410 in the Tc-99m TRODAT-1 target image in FIG. 4. The standard brain template establish from number of overlay normal brain images to show the putamen relative position 310 in the brain.

Reference is made to FIG. 5, FIG. 5 is a nuclear medicine image without the striatum region. The processing unit 120 further divides the putamen and the caudate nucleus of the striatum region to generate a reference region. A background value is calculated by the pixel value of the remainder region.

The processing unit 120 divides the striatum region in the scan image to consider as excluding both sides of striatum in the target image to obtain a reference region and calculating the background value based on the 75 percentile of the pixel intensity value in the reference region. Reference is made to FIG. 5, a red region presents the reference region of extracting whole brain without striatum region. The background value is calculated by the 75 percentile of the pixel intensity value in the reference region.

The computing unit 130 calculates a specific uptake ratio by subtracting the background value from the average pixel value then divided by the background value based on the formula

$$SUR = \frac{Target - Background}{Background}$$

An asymmetric index (ASI) is used to observe an asymmetric ratio of the two sides of striatum and the difference in uptake ratio between the two sides of striatum. The computing unit 130 calculates the asymmetry index based on an absolute value of the difference between the specific uptake ratio of the left side brain and the specific uptake ratio of the right side brain then divided by an average pixel value of two specific uptake ratios based on the formula

$$ASI = \frac{\vert {SUR}_{ipsilateral} - {SUR}_{contralateral} \vert}{( {SUR}_{ipsilateral} + {SUR}_{contralateral} ) / 2} \times 100 \%$$

wherein the SURᵢₚₛᵢₗₐₜₑᵣₐₗ is the specific uptake ratio of the left side brain; SUR_{contralateral} is the specific uptake ratio of the right side brain.

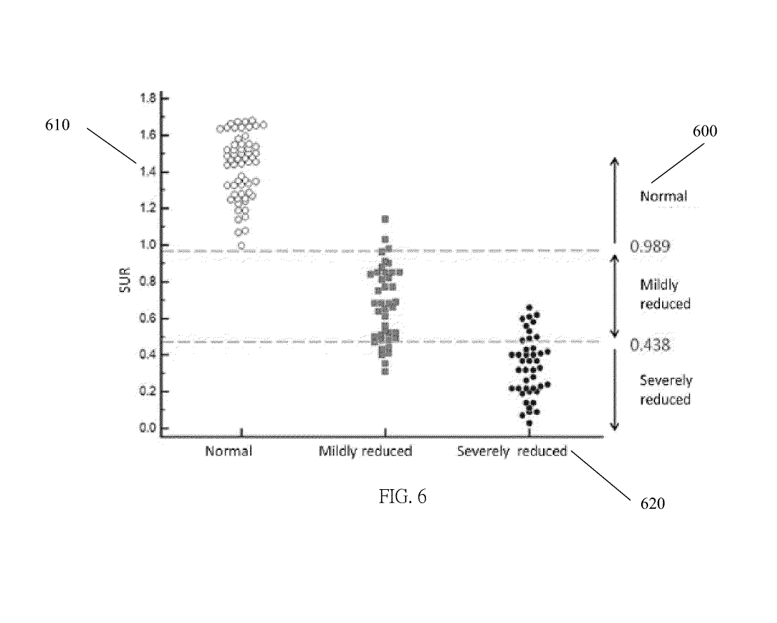

The processing unit 120 determines a reduced type of the striatum region is based on the specific uptake ratio and the asymmetry index. Reference is made to FIG. 6, which depicts a reduced type of the striatum. The display device 140 may be coupled to the computing device 130, which is configured to display the reduced type of the striatum based on a receiver operating characteristic (ROC), or ROC curve, that illustrates the reduced type of the striatum. The ROC curve can be generated by plotting the reduced type of the striatum of the specific uptake ratio in the y-axis 610 versus the reduced type probability in x-axis 620. As shown in FIG. 6, the analysis chart 600 illustrates three different reduced types, which includes normal, mildly reduced and severely reduced for the scan image. The striatum is identified as normal when the specific uptake ratio is over 0.989. The striatum is identified as mildly reduced when the specific uptake ratio is between 0.438 and 0.989. The striatum is identified as severely reduced when the specific uptake ratio is between 0 and 0.438.

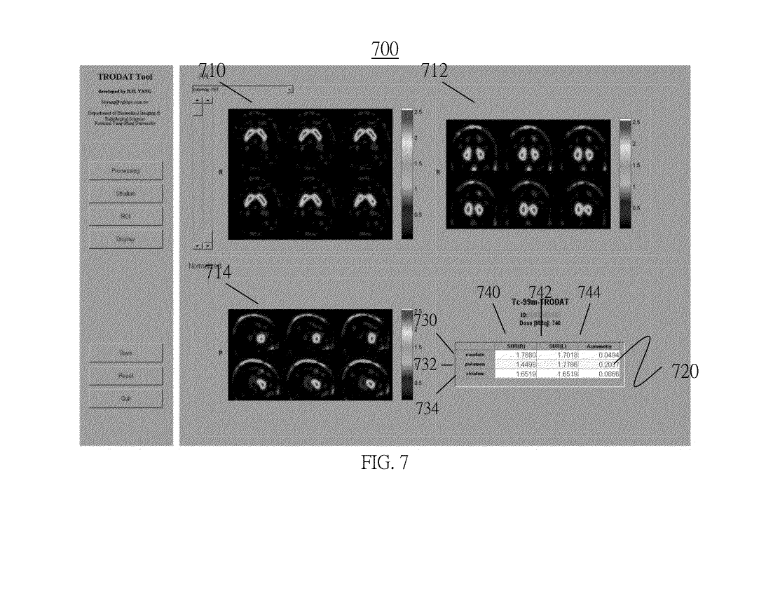

In the example of FIG. 7, FIG. 7 is a window of the system to process the nuclear medicine image. The display device 140 displays an analysis result of the scan image according to brain dopamine transporter binding with Tc-99m TRODAT-1. The display device 140 displays an interface 700 for quantitative analysis of nuclear medicine brain image. The interface 700 provides three different angles scan images 710, 712, 714 and an analysis chart 720. The analysis chart 720 comprises the specific uptake ratio of the right side of the brain (SUR(R)) 740 and the specific uptake ratio of the left side of the brain (SUR(L))742 and the asymmetry 744. The analysis chart 720 further comprises the caudate 730, the putamen 732 and the striatum 734.

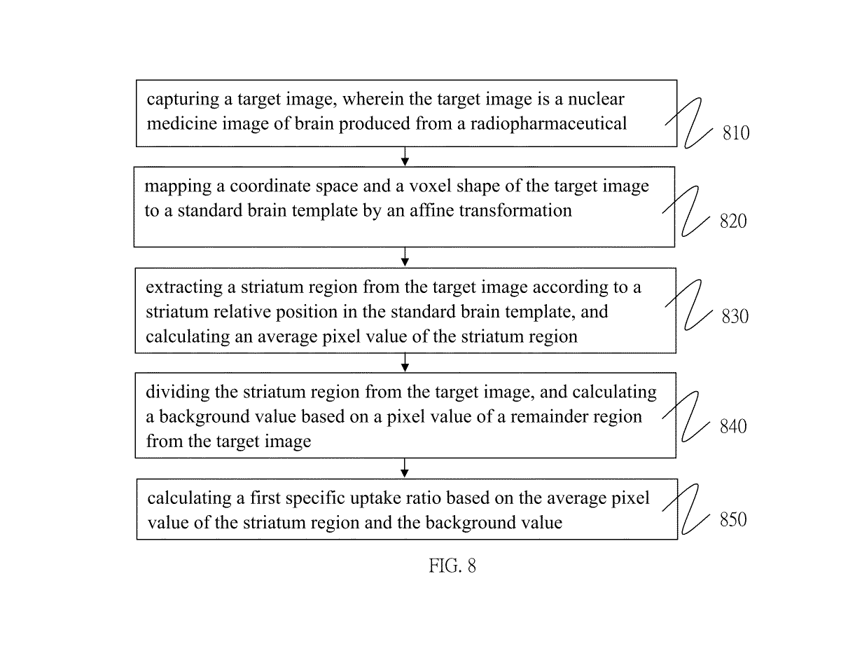

Reference is made to FIG. 8, which is a flowchart 800 in accordance with the method for quantitative analysis of nuclear medicine imaging performed by the system 100 of FIG. 1.

Beginning with block 810, the single photon emission computed tomography is performed by using a gamma camera to acquire multiple 2-D images (also called projections), from multiple angles. In particular, SPECT is used to obtain images of a γ-emitter distribution after its administration in the human body. The target images are obtained given a set of their projections, acquired using rotating gamma cameras. During SPECT acquisition, gamma detectors rotate around the patient usually over 180 or 360 degree. These target images are viewed in three orthogonal planes (transaxial, sagittal, and coronal). The target images show the radioactive tracer, for example Tc-99m TRODAT-lused to track the distribution of a substance within the brain tissue. Parkinson's disease occurs when nerve cells, or neurons, in an area of the brain that controls movement become impaired and/or die. Normally, these neurons produce an important brain chemical known as dopamine, but when the neurons die or become impaired, they produce less dopamine. This shortage of dopamine causes the movement problems of people with Parkinson's. The present invention provides a method and system based on characteristic of brain dopamine transporter binding with Tc-99m TRODAT-1 to help rule out the possibility of Parkinson's disease as early as possible by diagnosis of dopamine disorders.

In block 820, the processing unit 120 performs spatial normalization. The first step is to reshape an individual's brain to match the shape and size of a standard template image. This is a crucial step required for group-level statistical analyses. Spatial normalization is an image processing step, more specifically an image registration method. Human brains differ in size and shape, and one goal of spatial normalization is the spatial transformation of brains into a common space, making them comparable to each other. Human brains are variable in their size and shape. Such structural variability of brains imposes obstacles on intersubject brain function studies in how to determine regional correspondence from brain to brain despite of their divergence. The attempts are mainly focusing on setting up a reference frame in a three-dimensional Cartesian coordinate space as a common space for different brains to align to. The ultimate goal of spatial normalization is the spatial transformation of brains into a common space, making them comparable to each other. In final Talairach transformation step, each of the 12 sub-cuboids is compensated to match the corresponding a standard Talairach template (Talairach Daemon space) by mathematical stretching, squeezing and warping the sub-cuboids. The processing unit 120 (FIG. 1) maps a coordinate space, and a voxel shape of the target image to the standard brain template by an affine transformation, according to such mapping process, the difference between the target image and the standard Talairach template obtains minimum. Therefore, a voxel activity value of each sample is not modified too much and the original signal is not decreased or increased.

The processing unit 120 performs spatial normalization. In the second step, the shape of the brain is processed by nonlinear warping to match with the standard brain template. The ultimate goal of spatial normalization is the spatial transformation of brains into a common space. It builds up the nonlinear deformation fields based on linear combinations of smooth basis functions, wherein the basis functions is transformed from three-dimensional discrete cosine. The purpose of the first and second step is to define a minimum difference value between the original image and the standard brain template and optimizes the image.

In block 830, the processing unit 120 determines the striatum region in the target image based on the striatum relative position in the standard brain template and calculates the average pixel value of the striatum region. The processing unit 120 (FIG. 1) compares the Tc-99m TRODAT-1 target image with the standard brain template defined in the standard stereotactic space of the Automated Anatomical Labeling to determine the striatum region 410 in the Tc-99m TRODAT-1 target image. The processing unit 120 (FIG. 1) further calculates the average pixel value based on the striatum region 410 in the Tc-99m TRODAT-1 target image, wherein the striatum 310, 410 comprises the caudate nucleus and the putamen.

For example, the processing unit 120 divides the region of the caudate nucleus and the putamen in the Tc-99m TRODAT-1 target image corresponding to the region of the caudate nucleus and the putamen in standard brain template. The processing unit 120 further calculates the average pixel value based on the caudate nucleus and the putamen in the Tc-99m TRODAT-1 target image.

In 840 block, the striatum region in the target image is divided and a background value is calculated based on the pixel value of the reference region. The processing unit 120 extracts whole brain without the striatum region in the target image to generate a reference region and calculates the background value based on the 75th percentile of the pixel intensity value in the reference region 510. Reference is made to FIG. 5, the red region presents the reference region which extracted whole brain without the striatum region, the background value is calculated based on the 75th percentile of the pixel intensity value in the red region.

Reference is made to FIG. 4, the red region presents the reference region which extracted whole brain without the striatum region. The background value is calculated based on the 75 percentile of the pixel intensity value in the red region.

In 850 block, the specific uptake ratio is calculated based on the average pixel value and the background value. The computing unit 130 calculates the specific uptake ratio by subtracting the background value 510 from the average pixel value of the striatum region 410 then divided by the background value. A higher specific uptake ration is calculated in a particular region which presenting in the particular region has a higher uptake ratio than the reference region. The SURᵢₚₛᵢₗₐₜₑᵣₐₗ is the specific uptake ratio of the left side of the brain; SUR_{contralateral} is the specific uptake ratio of the right side of the brain.

The computing unit 130 calculates the asymmetric index based on an absolute value of the difference between the specific uptake ratio of the left side of the brain and the specific uptake ratio of the right side of the brain and divided by an average pixel value of two specific uptake ratios. The asymmetric index is used to observe an asymmetric ratio of the two sides of striatum and the difference in uptake ratio between the two sides of striatum.

The present invention provides an analysis result of the scan image according to brain dopamine transporter binding with Tc-99m TRODAT-1. The display device 140 displays an interface 700 for quantitative analysis of nuclear medicine brain image. The interface 700 provides three different angles scan images 710, 712, 714 and an analysis chart 720. The analysis chart 720 provides quantitative indexes that comprise the specific uptake ratio (SUR(R) and SUR(L)) and the asymmetry index for the caudate 730, the putamen 732 and the striatum 734 to observe the difference in the specific uptake ratio of the striatum on a three-dimensional surface for automatically determining Parkinson's disease stage. The present invention improves issues in time-consuming from analysis of manually selected regions, reproducibility and personal subjectivity and provides a reliable, objective and convenient tools for assessing Parkinson's disease.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

What is claimed is:

1. A method for quantitative analysis of nuclear medicine brain imaging, comprising:
receiving a target image, wherein the target image is a nuclear medicine image of brain produced from a radiopharmaceutical;
mapping a coordinate space and a voxel shape of the target image to a standard brain template by an affine transformation, wherein the standard brain template comprises a striatum relative position;
extracting a striatum region from the target image according to the striatum relative position in the standard brain template, and calculating an average pixel value of the striatum region;
dividing the striatum region from the target image, and calculating a background value based on a pixel value of a remainder region from the target image; and
calculating a first specific uptake ratio based on the average pixel value of the striatum region and the background value.

2. The method claim 1, wherein dividing the striatum region from the target image comprises:
extracting whole brain without the striatum region in the target image to generate a reference region; and
calculating the background value based on the 75 percentile of the pixel intensity value in the reference region.

3. The method claim 1, wherein the standard brain template comprises a putamen relative position and a caudate nucleus relative position of striatum.

4. The method claim 1, wherein the specific uptake ratio is calculated by subtracting the background value from the average pixel value then divided by the average pixel value.

5. The method claim 1, wherein the specific uptake ratio comprises a specific uptake ratio of the left side brain and a specific uptake ratio of the right side brain.

6. The method claim 5, wherein further comprises calculating an asymmetry index based on an absolute value of the difference between the specific uptake ratio of the left side brain and the specific uptake ratio of the right side brain then divided by an average pixel value of the specific uptake ratios based on the formula:
$$ASI = \frac{\vert {SUR}_{ipsilateral} - {SUR}_{contralateral} \vert}{( {SUR}_{ipsilateral} + {SUR}_{contralateral} ) / 2} \times 100 \%$$
wherein the SUPᵢₚₛᵢₜₐₜₑᵣₐₗ is the specific uptake ratio of the left side brain; SUR_{contralateral} is the specific uptake ratio of the right side brain.

7. The method claim 1, wherein the radiopharmaceutical is Tc-99m TROD AT-1.

8. The method claim 1, wherein determining a reduced type of the striatum region is based on the specific uptake ratio and the asymmetry index.

9. The method claim 1, wherein the target image is a scan imaging.

10. A system for quantitative analysis of nuclear medicine imaging, comprising:
an image capturing unit configured to capture a target image, wherein the target image is a brain image as a nuclear medicine image of brain produced from a radiopharmaceutical;
a processing unit configured to map a coordinate space and a voxel shape of the target image to a standard brain template by an affine transformation, wherein the standard brain template comprises a striatum relative position, then extract a striatum region from the target image according to the striatum relative position in the standard brain template, and calculate an average pixel value of the striatum region, dividing the striatum region from the target image, and calculating a background value based on a pixel value of a remainder region from the target image; and
a computing unit configured to calculate a first specific uptake ratio based on the average pixel value of the striatum region and the background value.

11. The system of claim 10, wherein the processing unit is further configured to extract whole brain without the striatum region in the target image to generate a reference region; and calculate the background value based on the 75 percentile of the pixel intensity value in the reference region.

12. The system of claim 10, wherein the standard brain template comprises a putamen relative position and a caudate nucleus relative position of striatum.

13. The system of claim 10, wherein the computing unit is further configured to calculate the specific uptake ratio by subtracting the background value from the average pixel value then divided by the average pixel value.

14. The system of claim 10, wherein the specific uptake ratio comprises a specific uptake ratio of the left side brain and a specific uptake ratio of the right side brain.

15. The system of claim 10, wherein the computing unit is further configured to calculate an asymmetry index based on an absolute value of the difference between the specific uptake ratio of the left side brain and the specific uptake ratio of the right side brain then divided by an average pixel value of the specific uptake ratios based on the formula:
$$ASI = \frac{\vert {SUR}_{ipsilateral} - {SUR}_{contralateral} \vert}{( {SUR}_{ipsilateral} + {SUR}_{contralateral} ) / 2} \times 100 \%$$
where the SURᵢₚₛᵢₗₐₜₑᵣₐₗ is the specific uptake ratio of the left side brain; SUR_{contralateral} is the specific uptake ratio of the right side brain.

16. The system of claim 10, wherein he radiopharmaceutical is Tc-99m TRODAT-1.

17. The system of claim 10, wherein the computing unit is further configured to determine a reduced type of the striatum region is based on the specific uptake ratio and the asymmetry index.

18. The system of claim 10, wherein further comprises a display device configured to display an interface for quantitative analysis of nuclear medicine brain image.

19. The system of claim 18, wherein the interface for quantitative analysis of nuclear medicine brain image is further configured to display the specific uptake ratio of the left side brain, the specific uptake ratio of the right side brain and the asymmetry index.

20. The system of claim 10, wherein the processing unit is further configured to divide a putamen and a caudate nucleus of the striatum region in the target image based on the standard brain template.
